(19) United States
(12) Patent Application Publication
(10) Pub. No.: US 2026/0199564 A1
(43) Pub. Date: Jul. 16, 2026

(54) METHOD OF PREVENTING BLOOD COAGULATION AND DRUG-ELUTING IMPLANT FOR PERCUTANEOUS CORONARY INTERVENTION

(71) Applicant: Sheau-Long LEE, Taoyuan City (TW)

(72) Inventors: Sheau-Long LEE, Taoyuan City (TW); Yu-Chieh LEE, Taoyuan City (TW); Wan-Han HSU, Taoyuan City (TW); Hsiang-Lung WANG, Taipei City (TW)

(73) Assignee: Sheau-Long LEE, Taoyuan City (TW)

(21) Appl. No.: 19/138,266

(22) PCT Filed: Feb. 22, 2024
(86) PCT No.: PCT/CN2024/078068

§ 371 Date: Jun. 12, 2025

Related U.S. Application Data
(60) Provisional application No. 63/486,287, filed on Feb. 22, 2023.

(51) Int. Cl. A61L 31/16; A61F 2/82; A61L 31/04; A61L 31/06

(52) U.S. Cl. CPC A61L 31/16; A61F 2/82; A61L 31/048; A61L 31/06; A61F 2210/0076; A61F 2250/0067; A61L 2300/42

(57) ABSTRACT

The present disclosure provides a method of preventing blood coagulation, comprising administering an effective amount of a ginsenoside Compound K (CK) to a subject in need. The present disclosure also provides use of a ginsenoside Compound K (CK) in the manufacture a medicament for preventing blood coagulation. Further, the present disclosure provides a drug-eluting percutaneous coronary intervention (PCI), comprising a blood vessel implant, wherein a surface of the blood vessel implant is coated with a first layer comprising ginsenoside Compound K (CK) and a first bioabsorbable polymer, wherein the first bioabsorbable polymer comprises Poly-L-lactic acid (PLLA) and Poly (L-Lactide-co-ε-Caprolactone) (PLCL) in a weight ratio of 60%-80%:20%-40%.

### BACKGROUND OF THE INVENTION

Coronary stents are medical devices that help treat patients with severe coronary artery disease. And the stents are usually being used along with balloon angioplasty surgery. Briefly, the balloon angioplasty will be performed to open the narrow arteries, and the stents will be placed in the vessels for support.

In the past, the incidences of stent thrombosis and in-stent restenosis within one month after installing traditional coronary stents are 1% and 15-20%, respectively. However, after the invention of Drug-eluting stents (DES) and Drug-eluting balloon (DEB), which can inhibit the growth of endothelial cells, the incidence of in-stent restenosis was significantly reduced to less than 1%. The drugs applied on DES and DEB include Paclitaxel, Sirolimus (also known as Rapamycin), and Limus-family drugs (such as Everolimus, Zotarolimus, and Billimus). These drugs can help inhibit the proliferation of endothelial cells and act as immunosuppressive agents, which will effectively reduce the risk of in-stent restenosis.

Although the current DES and DEB can solve the problem of restenosis, the patients will need to take long-term anticoagulants and live under the risk of late stent thrombosis. Besides, Paclitaxel and Sirolimus have been proved for high toxicity and low liposolubility, respectively.

On the other hand, spray temperature is an important issue, if the composition solvent under 100° C. will present high viscosity and block the chamber but we know the CK has much more solubility could overcome the disadvantage.

Therefore, it is necessary to discover new drugs applying to DES and DEB.

The previous techniques have not revealed the anticoagulation effect of the ginsenoside Compound K (CK) and the potential to act as an alternative drug applying on DES and DEB. In this invention, CK will be used on DES and DEB and help overcome the disadvantage of stent thrombosis and restenosis caused by current techniques.

Ginsenosides, the main active ingredients of ginseng, are known to have a variety of pharmacological activities, e.g. antitumor, antifatique, antiallergic and antioxidant activities. Ginsenosides share a basic structure, composed of gonane steroid nucleus having 17 carbon atoms arranged in four rings. Ginsenosides are metalized in the body, and a number of recent studies suggest that ginsenoside metabolites, rather than naturally occurring ginsenosides, are readily absorbed in the body and act as the active components. Among them, ginsenoside CK, also named Compound K (CK), is known as one metabolite of protopanaxadiol-type ginsenosides via the gypenoside pathway by human gut bacteria. Until now, no prior art references report the effect of ginsenoside CK in anticoagulation and its use for preparing coronary stents.

### SUMMARY OF THE INVENTION

In this case, CK is well-distributed on the surface of stents and the balloons (which are used in the balloon angioplasty) by the vacuum plasma spraying (VPS) technology. CK possesses the unique characteristics of low-toxic and antithrombotic; therefore, it would be a potential choice for clinical use in preventing stent thrombosis and restenosis.

In one aspect, the present disclosure provides a method of preventing blood coagulation, comprising administering an effective amount of a ginsenoside Compound K (CK) to a subject in need.

In another aspect, the present disclosure provides use of a ginsenoside Compound K (CK) in the manufacture a medicament for preventing blood coagulation.

In a further aspect, the present disclosure provides a ginsenoside Compound K (CK) for use in a method of preventing blood coagulation.

In still a further aspect, the present disclosure provides a drug-eluting percutaneous coronary intervention (PCI), comprising a blood vessel implant, wherein a surface of the blood vessel implant is coated with a first layer comprising ginsenoside Compound K (CK) and a first bioabsorbable polymer.

In one embodiment of the drug-eluting PCI, wherein the first bioabsorbable polymer comprises Poly-L-lactic acid (PLLA) and Poly(L-Lactide-co-ε-Caprolactone) (PLCL) in a weight ratio of 60%-80%:20%-40%.

In one embodiment of the drug-eluting PCI, wherein a thickness of the first layer ranging from 0.5 to 2 μm, from 0.5 to 1.5 μm, or from 0.8 to 1 μm.

In one embodiment of the drug-eluting PCI, wherein the CK in the first bioabsorbable polymer is 0.1 to 5 μg/mm², 0.1-3 μg/mm², 0.1-1 μg/mm², 0.25-0.75 μg/mm² or 1-3 μg/mm².

In one embodiment of the drug-eluting PCI, wherein the blood vessel implant is a stent.

In one embodiment of the drug-eluting PCI, further comprising a second layer on top of the first layer, wherein the second layer comprises a second bioabsorbable polymer, wherein the second bioabsorbable polymer comprises Polyvinylpyrrolidone (PVP) in a weight ratio of 80%-100% based on the total weight of the second bioabsorbable polymer.

In one embodiment of the drug-eluting PCI, wherein a thickness of the second layer ranging from 0.5 to 3 μm, from 1 to 3 μm, or from 0.5 to 1.5 μm.

In one embodiment of the drug-eluting PCI, further comprising a third layer between the first layer and the second layer, wherein the third layer comprises the CK and a third bioabsorbable polymer, wherein the third bioabsorbable polymer comprises PLLA and PLCL in a weight ratio of 25%-35%:65%-85%.

In one embodiment of the drug-eluting PCI, wherein a thickness of the third layer ranging from 0.5 to 1.5 μm.

In one embodiment of the drug-eluting PCI, wherein the CK in the third bioabsorbable polymer is 0.1 to 5 μg/mm², 0.5-2 μg/mm², preferably 0.75-1.25 μg/mm².

In one embodiment of the drug-eluting PCI, wherein the weight of the CK applied on the blood vessel implant is more than 100 μg and less than 1000 μg.

In one embodiment of the drug-eluting PCI, further comprising a second layer under the first layer, wherein the second layer comprises a second bioabsorbable polymer, wherein the second bioabsorbable polymer comprises Polyvinylpyrrolidone (PVP) in a weight ratio of 80%-100%.

In one embodiment of the drug-eluting PCI, wherein a thickness of the second layer ranging from 0.5 to 3 μm, from 1 to 3 μm, or from 0.5 to 1.5 μm.

In one embodiment of the drug-eluting PCI, wherein the first layer coated on the surface of the blood vessel implant is coated by the vacuum plasma spraying (VPS).

### BRIEF DESCRIPTION OF THE DRAWINGS

FIG. 1 shows the exemplary three-layer coating of ginsenoside CK on the stent.

FIG. 2 shows the releasing kinetics curve of the ginsenoside CK during the healing process. Nearly 80% of drug is released within one month (initial burst). Remaining drug is programmed to get released for 3 months. Designed to cover the entire period of arterial wound healing in real-world patients. After 90 days of drug release, the amount of residual drug in the stent is so low that it goes beyond the detection/quantitation limit.

FIG. 3 shows the exemplary two-layer coating of ginsenoside CK on the balloon.

FIG. 4 shows the releasing kinetics curve of the ginsenoside CK during the healing process. Nearly 80% of drug is released within one month (initial burst). Remaining drug is programmed to get released for 3 months. Designed to cover the entire period of arterial wound healing in real-world patients. After 90 days of drug release, the amount of residual drug in the stent is so low that it goes beyond the detection/quantitation limit.

FIG. 5 shows the inhibition curve of Astemizole using hERG FP assay. Data points represent the mean of three determinations.

FIG. 6 shows the inhibition curve of CK using hERG FP assay. Data Points represent the mean of three determinations.

FIG. 7 shows CK on platelet aggregation.

FIG. 8 shows that HUVECs were incubated with CK alone (1 μM, 3 μM, and 10 μM) for 24 h, and cell viability was assayed by the CCK-8 assay.

FIG. 9 shows the effect of ginsenoside CK on LPS-induced IL-6 and TNF-α expression in HUVECs. HUVECs were treated for 1 h with ginsenoside CK (1 μM, 3 μM, and 10 μM) prior to LPS (1 μg/ml) stimulation for 24 h (A) IL-6 (B) TNF-αexpression. Values are means±SEM of 3 independent experiments. Statistical significance assessed by one-way ANOVA followed by Scheffe post-hoc test for multiple comparisons (***P<0.01, *P<0.05 vs LPS).

FIG. 10 shows the effect of ginsenoside CK on LPS-induced ICAM-1 and VCAM-1 mRNA expression in HUVECs. HUVECs were treated for 1 h with ginsenoside CK (1 μM, 3 μM, and 10 μM) prior to LPS (1 μg/ml) stimulation for 24 h. (A) ICAM-1 (B) VCAM-1 mRNA expression. Values are means±SEM of 3 independent experiments. Statistical significance assessed by one-way ANOVA followed by Scheffe post-hoc test for multiple comparisons (***P<0.01, *P<0.05 vs LPS).

### DETAILED DESCRIPTION OF THE EMBODIMENTS

The foregoing and other aspects of the present disclosure will now be described in more detail with respect to other embodiments described herein. It should be appreciated that the invention can be embodied in different forms and should not be construed as limited to the embodiments set forth herein. Rather, these embodiments are provided so that this disclosure will be thorough and complete and fully convey the invention's scope to those skilled in the art.

The terminology used in the description of the invention herein is for the purpose of describing particular embodiments only and is not intended to be limiting to the invention. As used in the description of the invention and the appended claims, the singular forms “a”, “an” and “the” are intended to include the plural forms as well, unless the context clearly indicates otherwise.

As used herein, the terms “comprises,” “comprising,” “includes,” “including,” “has,” “having,” “contains”, “containing,” or any other variation thereof, are intended to cover a non-exclusive inclusion, subject to any limitation explicitly indicated. For example, a composition, mixture, process or method that includes a list of elements is not necessarily limited to only those elements but may include other elements not expressly listed or inherent to such composition, mixture, process, or method.

As used herein, the term “about” indicates that a value includes, for example, the inherent variation of error for a measuring device, the method employed to determine the value, or the variation among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer to alternatives only or the alternatives are mutually exclusive. However, the disclosure supports a definition that refers to only alternatives and “and/or.”

Unless otherwise defined, all technical and scientific terms used herein have the same meaning as commonly understood by one of ordinary skill in the art to which this invention belongs. All publications, patent disclosure, patents, and other references cited herein are incorporated by reference in their entirety for the teachings relevant to the sentence and/or paragraph in which the reference is presented.

The following representative examples and embodiments illustrate various features and embodiments of the disclosure, which are intended to be illustrative and not limiting. Those skilled in the art will readily appreciate that the specific examples are only illustrative of the invention as described more fully in the subsequent claims. Every embodiment and feature described in the present application should be understood to be interchangeable and combinable with every embodiment contained within.

### 1. Materials and Methods

### 1.1 Investigational Drugs

a. Ginsenoside compound K (CK) powder is provided by Wellhead Biological Technology Corp., Purity: 95%
b. Collagen (1×2 ml) (Helena Laboratories, Shang Li Instruments Co., Ltd)
c. Human platelets were separated from the blood samples before tests
d. Dimethyl Sulfoxide (Lot. #RNBH8393, Sigma)
e. 10× Phosphate Buffer Saline (Lot. AC13074270, HyClone)
f. Predictor™ hERG Fluorescence Polarization Assay kit (PV 5365, Invitrogen, CA, USA) was purchased from Invitrogen (Carlsbad, CA, USA).
g. The 384-well polypropylene plates (Corning #3657) and black 384-well assay plates (Corning #3677) were purchased from Corning (Lowell, MA, USA).

### 1.2 Experimental Equipment

a. 2.7 ml blood collection tubes (containing 3.2% sodium citrate)
b. Safety blood collection set with 21G luer adapters
c. Alcohol cotton balls
d. Tourniquets
e. 15-ml centrifuge tubes and 1.5-ml microcentrifuge tubes
f. 200- and 1000-μl plastic tips
g. 96-well microplates
h. Adjustable volume pipettes

### 1.3 Experimental Devices

a. Biosensors Technology—Auto-Pipette
b. Epoch™ Micro-Volume Spectrophotometer System (BioTek)
c. AggRAM analyzer (Helena Laboratories)
d. Evolution™ Spectrophotometers (Thermo Scientific™)
e. Tecan Infinite® F200 micro-plate reader equipped with a fluorescence polarization module was purchased from TECAN Group Ltd (Switzerland). A channel handheld disposable-tip pipettor (eight-tip head) was purchased from Axygen (CA, USA).

### 2. Experiment Operation and Operating Procedures

### CK-Coating on Stent

### 2.1 Preparation of CK-Coating on the Superflex Cruz

The composition of PLLA(80-95%) and PLCL(1-10%) mix well with chloroform under water heating, then add DMSO at 25° C. put the CK (concentration 0.5-1.5 μg/ml). Controlling the Spray chamber temperature below 100 T to avoid the polymer de-formulation at high temperature.

The detail step is below:

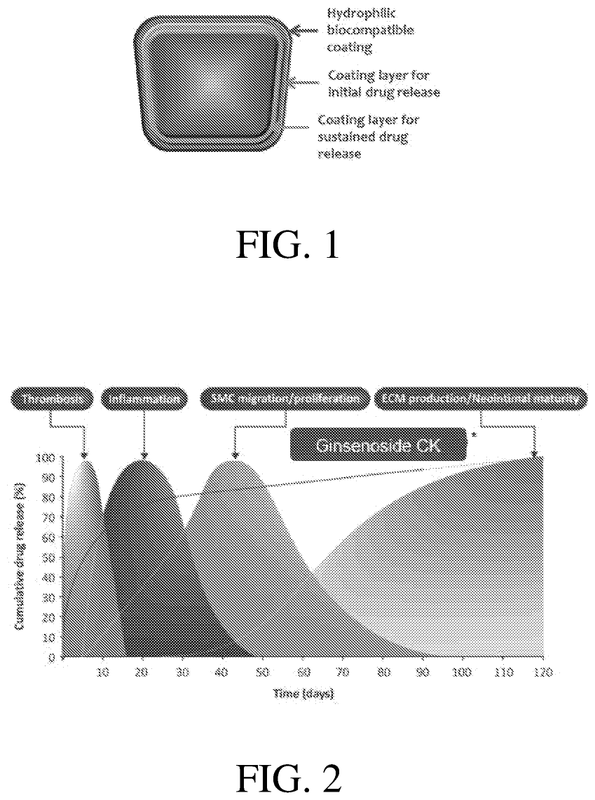

The CK DES has Conformal Coating. It has three-layer coating, as shown in FIG. 1, included as following:
Top Layer: Thickness is 0.5-1.5 μm. This layer is drug free polymeric layer incorporating hydrophilic PVP (Polyvinylpyrrolidone). Protects from light, moisture, premature drug release and provides lubricity as well during stent implantation.
Middle layer: Thickness is 0.5-1.5 μM. Same as base layer it contains blends of PLLA (Poly-L-lactic acid), PLCL (Poly(L-Lactide-co-ε-Caprolactone)) and CK drug (0.5-2 μg/mm²). Designed to provide sufficient amount of drug after stent implantation
Base layer: Thickness is 0.5-1.5 μm. This layer contains blends of PLLA (Poly-L-lactic acid), PLCL (Poly(L-Lactide-co-ε-Caprolactone)) and CK drug (0.1-1 μg/mm²). Programmed for Slow Drug Release.

### 2.2 Healing Process as Shown in FIG. 2.

The kinetics curve as indicated by arrow.

### 2.3 Coating Process

### 2.4 Polymer and Drug Preparing:

| | Polymer | Solvent | Drug |
|---|---|---|---|
| | Mixed Ratio | condition | Concentration |
| Top layer | PVP 80-100% | DMSO/25° C. | 0 |
| Middle layer | PLLA/PLCL: | DMSO/25° C. | 0.5-2 μg/mm² |
| | 25-35%/65-85% | | |
| Base layer | PLLA/PLCL: | DMSO/25° C. | 0.1-1 μg/mm² |
| | 60-80%/20-40% | | |

* Total CK DES the Drug Concentration is 0.5-2.5 μg/mm²
* Total CK DES per stent (3.0 mm × 20 mm) Drug is 250-350 μg

### CK-Coating on Balloon

### 2.5 Preparation of CK-Coating on the Balloon

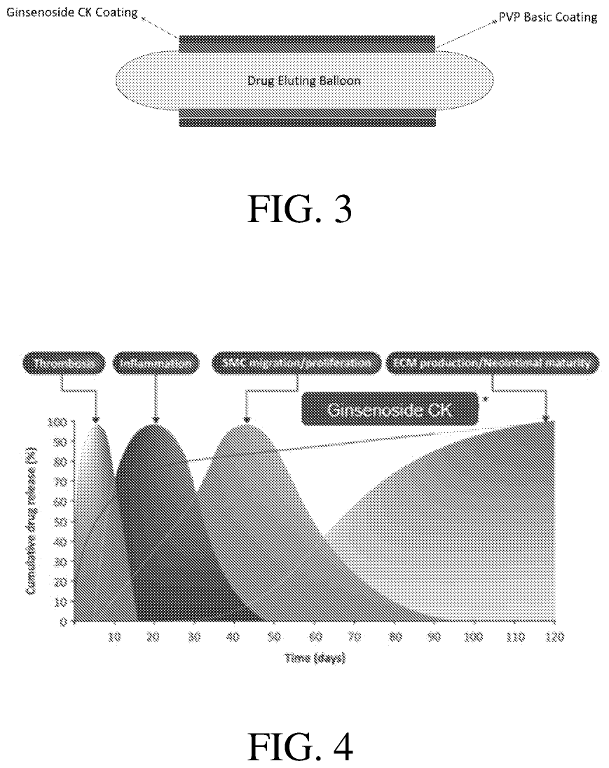

The CK DEB has Abluminal Coating. It has Two-layer coating included as shown in FIG. 3:
Top layer: Thickness is 1-3 μm. The layer it contains blends of PLLA (Poly-L-lactic acid), PLCL (Poly(L-Lactide-co-ε-Caprolactone)) and CK drug (2 μg/mm²). Designed to provide sufficient amount of drug after DEB implantation.
Base layer: Thickness is 0.5-2 μm. This layer is drug free polymeric layer incorporating hydrophilic PVP (Polyvinylpyrrolidone). Designed to provide the top layer bounding on balloon surface and peeling away after DEB implantation.

### 2.6 Healing Process as Following.

Drug Release Kinetics: Able to cover 120 days vessel healing process as shown in FIG. 4. The kinetics curve as indicated by arrow.

### 2.7 Coating Process

### Polymer and Drug Preparing:

| | Polymer | Solvent | Drug |
|---|---|---|---|
| | Mixed Ratio | condition | Concentration |
| Base layer | PVP 80-100% | DMSO/25° C. | 0 |
| Top layer | PLLA/PLCL: | DMSO/25° C. | 1-3 μg/mm² |
| | 60-80%/20-40% | | |

* Total CK DEB the Drug Concentration is 1-3 μg/mm²
* Total CK DEB per balloon (3.0 mm × 20 mm) Drug is 350-450 μg

hERG Assay

### 2.8 Preparation of Chemical Agents and Solution

a. Primary stock solutions of 3 mM CK and astemizole in 100% DMSO were diluted in 3-fold serial dilutions in the same solvent to obtain 16 different concentrations ranging from 3 mM to 210 μM. These solutions were further diluted 25-fold with FP (fluorescence polarization) assay buffer and then transferred to the assay plate.
b. Each test solution in the assay plate was further diluted 4-fold with FP assay buffer. Predictor™ hERG membrane preparations were thawed at room temperature and sonicated. Predictor™ hERG Tracer Red (250 nM) was diluted to 4 nM with hERG FP assay buffer. All experiments were performed in FP assay buffer containing 5% DMSO.

### 2.9 Experimental Procedures

a. The FP assay was carried out using Predictor™ hERG Fluorescence Polarization Assay Kit. The binding assay was conducted following the manufacturer's recommended protocol with some modifications. Aliquots (5 μL) of each concentration of test article of reference were pipetted into the appropriate wells of a black 384-well micro-plate containing 10 μL of hERG membrane and 5 μL of 4 nM fluorescent tracer and covered with plate lid to protect the reagents exposed to from light.
b. The experiments were performed using triplicate wells for each concentration. Following a 3-hour incubation at room temperature, the microplates were read on a Tecan Infinite® F200 plate reader using polarized excitation filter setting at 535 nm and emission filter setting at 590 nm (25 and 20 nm bandwidths, respectively). Assay robustness was assessed by measuring 16 replicates positive control wells (containing 30 μM E-4031, known hERG channel blocker, in FP assay mixture) and negative control wells (no hERG channel blocker in FP assay mixture).

### 2.10 Determination of Assay Z′ Value

a. To evaluate the robustness of the hERG assays, the signal-to-noise ratio was quantified by calculating Z′-factor for each assay. In statistics, the Z′-factor provides a way to evaluate the quality of the assay. Z values of greater than 0.5 are generally considered to be good assay performance, while the value of 1 indicates a theoretically ideal assay with no variability. Z′-factor values were determined using the blank subtracted polarization values from 16 wells of
negative and positive controls using the formula of Zhang et al. [1]

$${Z}^{′} = \text{?} - \frac{3 {σ}_{\text{?}} \text{?} 3 {σ}_{\text{?}}}{\vert {μ}_{\text{?}} - {μ}_{\text{?}} \vert} \text{?}$$

$$\text{?} \text{indicates text missing or illegible when filed}$$

Where μ_{NC} and σ_{NC} are the mean and standard deviation for the negative control (pre-drug signals exposed to vehicle), and μ_{PC} and σ_{PC} are the mean and standard deviation for the positive control (post-drug signals exposed to 30 μM positive inhibitor), respectively.

### 2.11 Data Analyses

All data analysis and presentation was performed using Microsoft® (Redmond, WA, USA) Office Excel 2010 and SigmaPlot version 12 (SPSS, Inc., Chicago, IL). For the concentration-response curve analysis, inhibition data were fit to the Four Parameter Logistic Equation:

$$E = {E}_{\text{?}} - \frac{( {E}_{\text{?}} - {E}_{\text{?}} )}{1 + {( \frac{x}{{IC}_{\text{?}}} )}^{\text{?}}}$$

$$\text{?} \text{indicates text missing or illegible when filed}$$

E=response of fluorescence polarization (bound ligand in the presence of an inhibitor concentration)
E max=the maximum response of fluorescence polarization
E min=the minimal response of fluorescence polarization
x=the inhibitor concentration
IC₅₀=the concentration of drug required to inhibit current by 50%
Hillslope=characterizes the slope of the curve at its midpoint

The suggested risk-classification of potency for hERG blocker is listed in the following table:

| Risk Class | IC₅₀ | |
|---|---|---|
| High-potency | <0.1 | μM |
| Moderate-potency | 0.1-1 | μM |
| Low-potency | >1 | μM |

### Cytochrome P450 Inhibition in Human Liver Microsomes Using LC-MS/MS Analysis

In IC₅₀ shift experiments, CK (0.2 μL prepared in DMSO) was pre-incubated for 30 min at 37° C., at 8 different concentrations, with pooled human liver microsomes (0.1-1 mg/mL, depending on which enzyme was assessed) in the absence or presence of 1 mM NADPH in 100 μL of potassium phosphate buffer (100 mM, pH 7.4) and MgCl₂ (2.5 mM). After the pre-incubation, a substrate mixture containing a probe substrate in 100 μL of potassium phosphate buffer (100 mM, pH 7.4), MgCl₂ (2.5 mM), and NADPH (1 mM) was added for measurement of the corresponding P450 activities. Incubations were terminated by addition of 200 μL methanol. Samples were then extracted and centrifuged and the supernatant was subjected to validated LC-MS/MS analysis.

### Anticoagulant Test

### 2.12 Preparation of Chemical Agents and Solution

a. A 20 mM CK stock solution is prepared with DMSO and is diluted with 1×PBS to the different working concentrations (0, 62.5, 125, 250, 500, 1000, and 2000 μM) before experiments.

### 2.13 Experimental Procedures

a. Blood Sample Collection

The donator should rest and fast for 12 h and not smoke before blood collection. Medication such as antihistamines, antibiotics, Aspirin, and anti-inflammatory drugs are not allowed to take 10-14 days before tests due to the potential of affecting platelet function.

To ensure the absence of coagulation, the blood sample should be fully mixed with the anticoagulant and stored at 24-27° C. It is recommended to conduct the platelet aggregation test within 30-150 min after blood sample collection.

b. Preparation of Platelet-Rich Plasma (PRP)

Slightly invert the blood collection tubes 3-4 times to resuspend the blood cells. Centrifuge the samples at 170×g for 7 min and transfer the supernatants to the new tubes. The samples should be used within 5-10 min after separation; otherwise, the samples should be stored at 4° C. to avoid agglutination.

c. Preparation of Platelet-Poor Plasma (PPP)

Slightly invert the blood collection tubes 3-4 times to resuspend the blood cells. Centrifuge the samples at 2,400×g for 10 min and transfer the supernatants to the new tubes. The samples should be tested within 5 min after being separated from whole blood; otherwise, the samples should be stored at 4° C.

d. Platelet Count

The concentration of PRP is diluted to 3×10⁸ cells/ml with PPP.

e. Platelet Aggregation Test

To test the effect of CK on the function of platelet aggregation, different concentrations of CK (0, 2.5, 5, 10, 20, 40, and 80 μM) are added to 0.25 ml PRP. The mixture samples are incubated at 37° C. for 2 minutes and immediately treated with 5 μg/ml collagen (100 μg/ml stock solution). The PRP-DMSO-distilled water mixture is used as non-aggregated control. For blank control groups, (1) the original PPP, (2) the PPP-DMSO-distilled water mixture, (3) the PPP-DMSO-collagen mixture, and (4) the PPP-CK-collagen mixture is prepared. All the mixture samples and control groups are incubated at 37° C. with an orbital shaker at 200 rpm for 5 min. Samples are then centrifuged at 170×g for 7 min and 200 μl of supernatants are transferred to a 96-well microplate. OD value is measured with a spectrometer at 650 nm, and the value is converted into an aggregate percentage.

| Experiment group | | 1 | 2 | 3 | 4 | 5 | 6 | 7 | 8 | 9 |
|---|---|---|---|---|---|---|---|---|---|---|
| PRP | CK(μM) | 0 | [symbol] | [symbol] | [symbol] | [symbol] | [symbol] | [symbol] | [symbol] | [symbol] |
| | DMSO | — | — | — | — | [symbol] | — | [symbol] | — | — |
| | Collagen | — | — | — | — | [symbol] | — | [symbol] | — | — |

| | | | |
|---|---|---|---|
| distilled water | — | — | — |

[symbol]

indicates data missing or illegible when filed

| | | | | |
|---|---|---|---|---|
| Blank group | 1 | 2 | 3 | 4 |

| | | | | | |
|---|---|---|---|---|---|
| PPP | CK | — | — | — | — |
| | DMSO | — | — | — | — |

| | | | |
|---|---|---|---|
| Collagen | — | — | — |
| distilled water | — | — | — |

### 2.14 Data Analyses

The aggregation percentage of platelet is calculated with the following formula:

$$a . Palatelet aggregation ( \% ) of Group 1 = 100 \% - [ \frac{PRP - PPP}{PRP - PPP} \times 100 \% ]$$

$$b . Platelet aggregation ( \% ) of Group 2 = 100 \% - [ \frac{\text{?}}{\text{?}} \times 100 \% ]$$

$$c . Platelet aggregation ( \% ) of Group 3 - 9 = 100 \% - [ \frac{\text{?}}{\text{?}} \times 100 \% ]$$

$$\text{?} \text{indicates text missing or illegible when filed}$$

### 3. Results and Discussion

hERG Assay

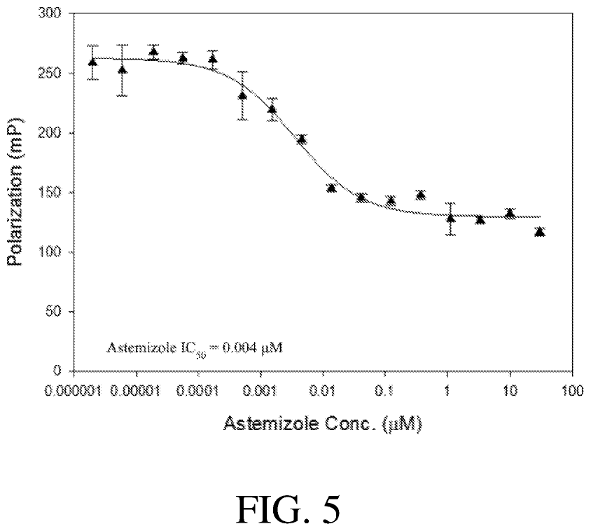

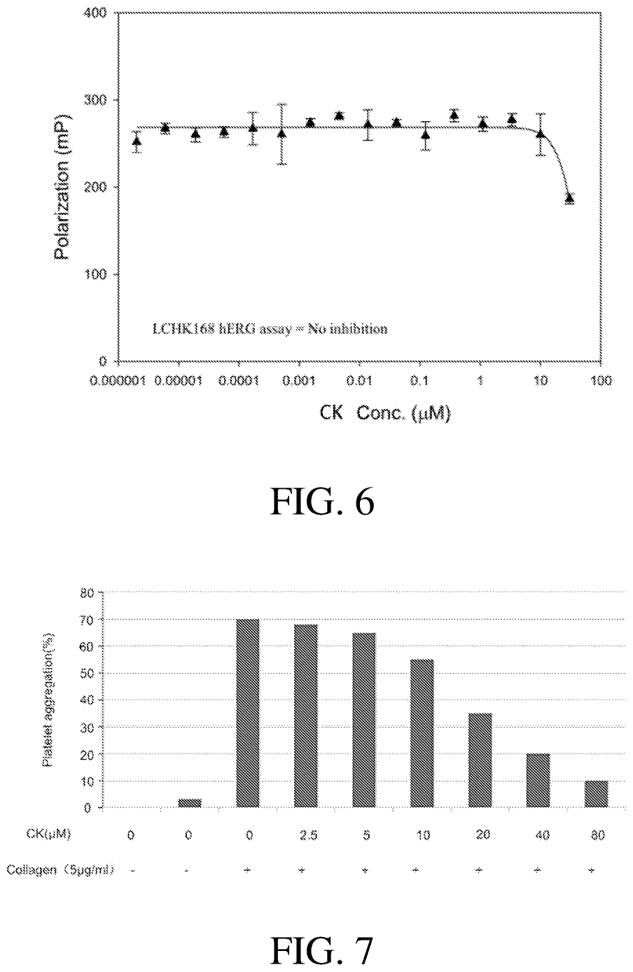

The hERG IC₅₀ value of astemizole (reference compound) was 0.004 μM from triplicate measurements. FIG. 5 shows log concentration-response curve for inhibitor astemizole. This IC₅₀ value of astemizole was well in agreement with those presented in the literature with this method. CK demonstrates minor inhibition in fluorescence polarization assay (FIG. 6). CK showed minor blocking effect for binding of tracer to the hERG membrane protein at 30 μM (from triplicate measurements) and the IC₅₀ value of CK was reported as >30 μM. CK could be classified as a weak ligand (IC₅₀>1 μM) for hERG channel (Table 1).

TABLE 1
Summary of IC₅₀ Values of CK and Astemizole

| Compound Name | Used as | IC₅₀ (μM) |
|---|---|---|
| CK | Test article | >30 |
| Astemizole | Assay control | 0.004* |

*IC₅₀ value of astemizole is in good agreement with the IC₅₀ value reported in the literature.

### Cytochrome P450 Inhibition in Human Liver Microsomes Using LC-MS/MS Analysis

Mean IC₅₀ values of inhibitor CK for of eight CYP-specific probe substrates of CYP1 A2, 2B6, 2C8, 2C9, 2C19, 2D6 and 3A4 in pooled human liver microsomes were >50, 14.84, >50, >50, >50, >50, 9.76 (midazolam as CYP3A4 substrate) and 13.86 (testosterone as CYP3A4 substrate) μM (Table 2), respectively. CK exhibited an IC₅₀ shift of 1.08, 0.98 and 1.06-fold with a 30 min pre-incubation for CYP2B6, 3A4 (midazolam as substrate) and 3A4 (testosterone as substrate) inhibition assay, respectively. CK did not inhibit CYP1A2, 2C8, 2C9, 2C19 and 2D6 at concentration of 50 μM following a 30 min pre-incubation in the presence or absence of NADPH (Table 3); therefore, no meaningful IC₅₀ shift values could be calculated for these isoforms.

TABLE 2
IC₅₀ Values of Inhibitor CK for Seven Human Cytochrome
P450 Enzymes in Pooled Human Liver Microsomes

| Enzyme | Substrate | Inhibitor | IC₅₀ (μM) * |
|---|---|---|---|
| CYP1A2 | Phenacetin | CK | >50 |
| CYP2B6 | Bupropion | CK | 14.84 |
| CYP2C8 | Amodiaquine | CK | >50 |
| CYP2C9 | Diclofenac | CK | >50 |
| CYP2C19 | S-Mephenytoin | CK | >50 |
| CYP2D6 | Dextromethorphan | CK | >50 |
| CYP3A4 | Midazolam | CK | 9.76 |
| CYP3A4 | Testosterone | CK | 13.86 |

* When IC₅₀ was greater than 50 μM, the percent inhibition value at 50 μM was included.

TABLE 3
IC₅₀ Values for CK of Human P450 Enzymes Following
a 30 min Pre-incubation with Human Liver Microsomes
in the Absence and Presence of NADPH

| | | | | IC₅₀ |
|---|---|---|---|---|
| | | IC₅₀ (μM) of | | shift^{a} |
| | | 30 min pre-incubation | | (fold |
| Inhibitor | Enzyme | [symbol] NADPH | [symbol] NADPH | difference) |
| LCHK168 | CYP1A2 | >50^{b} | >50^{b} | ND^{c} |
| | CYP2B6 | 12.81 | 11.90 | 1.08 |
| | CYP2C8 | >50^{b} | >50^{b} | ND^{c} |
| | CYP2C9 | >50^{b} | >50^{b} | ND^{c} |
| | CYP2C19 | >50^{b} | >50^{b} | ND^{c} |
| | CYP2D6 | >50^{b} | >50^{b} | ND^{c} |
| | CYP3A4(M) | 11.68 | 11.92 | 0.98 |
| | CYP3A4(T) | 16.73 | 15.74 | 1.06 |

^{a}The ratio of IC₅₀ with −NADPH in the pre-incubation compared to IC₅₀ with +NADPH in the pre-incubation
^{b}When IC₅₀ was greater than 50 μM, the percent inhibition value at 50 μM was included
^{c}ND, not determined [symbol]
indicates data missing or illegible when filed

### Anticoagulant Test

The experiment aims to unclear the effect of CK on platelet aggregation. After treating the platelet with CK, the aggregation level is decreased. Furthermore, the platelet aggregation level of group 9 is roughly 7-times less than group 4. These results indicate that CK can act as an anticoagulant to the platelets (FIG. 7).

### Effect of Ginsenoside CK on LPS-Induced IL-6 and TNF-α Expression in HUVECs

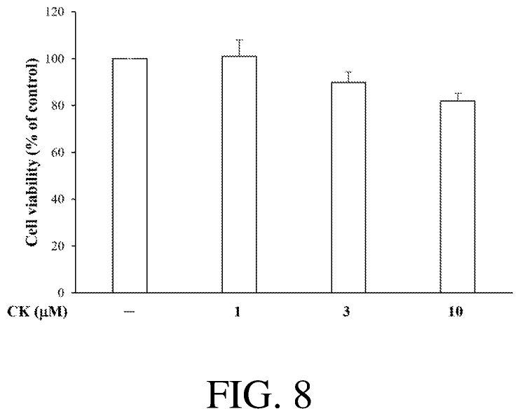

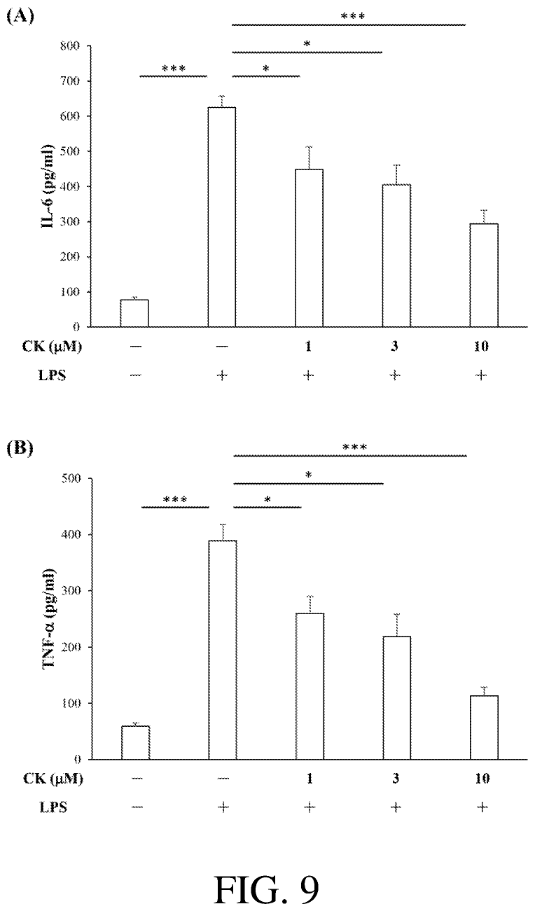

The results revealed that treatments with various concentrations (1 μM, 3 μM, and 10 μM) of ginsenoside CK alone for 24 h did not influence cell viability, indicating the nontoxicity of ginsenoside CK (FIG. 8). Treatment of HUVECs with LPS (1 μg/ml) increased IL-6 and TNF-αexpression. To determine whether LPS-induced IL-6 and TNF-αexpression was affected by ginsenoside CK, HUVECs were treated for 1 h with ginsenoside CK (1 μM, 3 μM, and 10 μM) prior to LPS (1 μg/ml) stimulation for 24 h. Ginsenoside CK significantly decreased LPS-induced IL-6 (FIG. 9, part A) and TNF-α (FIG. 9, part B) expression in a concentration-dependent manner.

Effect of Ginsenoside CK on LPS-Induced ICAM-1 and VCAM-1 mRNA Expression in HUVECs

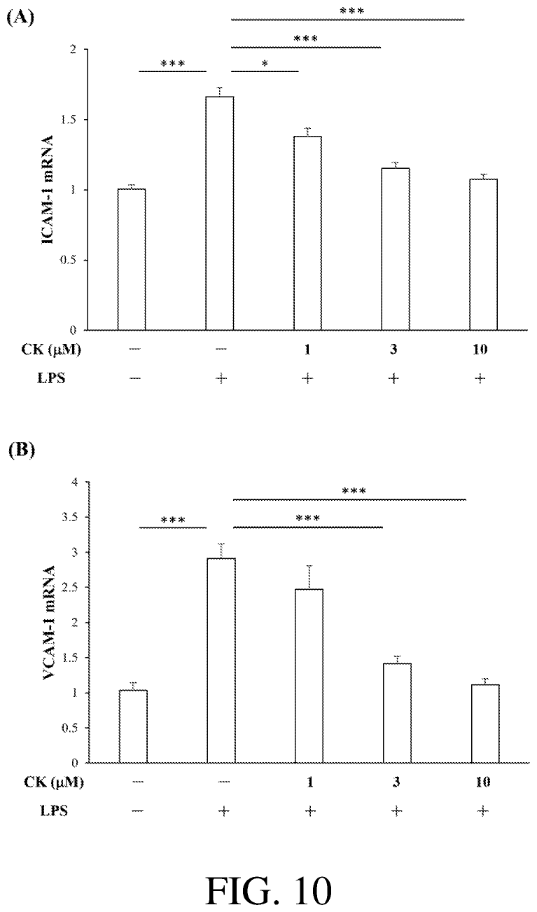

Treatment of HUVECs with LPS (1 μg/ml) increased ICAM-1 and VCAM-1 mRNA expression. Twenty-four hours after LPS treatment, maximal expression of ICAM-1 and VCAM-1 was attained. To determine whether LPS-stimulated ICAM-1 and VCAM-1 mRNA expression is affected by ginsenoside CK, HUVECs were treated for 1 h with ginsenoside CK (1 μM, 3 μM, and 10 μM) prior to LPS (1 μg/ml) stimulation for 24 h. Ginsenoside CK significantly inhibited ICAM-1 (FIG. 10, part A) and VCAM-1 (FIG. 10, part B) mRNA expression stimulated with LPS in a concentration-dependent manner.

The invention is now described and further exemplified by the following embodiments:
Embodiment 1. A drug-eluting percutaneous coronary intervention (PCI), comprising a blood vessel implant,
wherein a surface of the blood vessel implant is coated with a first layer comprising ginsenoside Compound K (CK) and a first bioabsorbable polymer.
Embodiment 2. The drug-eluting PCI of Embodiment 1, wherein the first bioabsorbable polymer comprises Poly-L-lactic acid (PLLA) and Poly(L-Lactide-co-ε-Caprolactone) (PLCL) in a weight ratio of 60%-80%:20%-40%.
Embodiment 3. The drug-eluting PCI of Embodiments 1 and 2, wherein a thickness of the first layer ranging from 0.5 to 2 μm, from 0.5 to 1.5 μm, or from 0.8 to 1 μm.
Embodiment 4. The drug-eluting PCI of any one of Embodiments 1 to 3, wherein the CK in the first bioabsorbable polymer is 0.1 to 5 μg/mm², 0.1-3 μg/mm², 0.1-1 μg/mm², 0.25-0.75 μg/mm² or 1-3 μg/mm².
Embodiment 5. The drug-eluting PCI of any one of Embodiments 1 to 4, wherein the blood vessel implant is a stent.
Embodiment 6. The drug-eluting PCI of any one of Embodiments 1 to 5, further comprising a second layer on top of the first layer,
preferably the second layer comprises a second bioabsorbable polymer, more preferably the second bioabsorbable polymer comprises Polyvinylpyrrolidone (PVP) in a weight ratio of 80%-100% based on the total weight of the second bioabsorbable polymer.
Embodiment 7. The drug-eluting PCI of any one of Embodiments 1 to 6, wherein a thickness of the second layer ranging from 0.5 to 3 μm, from 1 to 3 μm, or from 0.5 to 1.5 μm.
Embodiment 8. The drug-eluting PCI of any one of Embodiments 1 to 7, further comprising a third layer between the first layer and the second layer,
preferably the third layer comprises the CK and a third bioabsorbable polymer, more preferably the third bioabsorbable polymer comprises PLLA and PLCL in a weight ratio of 25%-35%:65%-85%.
Embodiment 9. The drug-eluting PCI of any one of Embodiments 1 to 8, wherein a thickness of the third layer ranging from 0.5 to 1.5 μm.
Embodiment 10. The drug-eluting PCI of any one of Embodiments 1 to 9, wherein the CK in the third bioabsorbable polymer is 0.1 to 5 μg/mm², 0.5-2 μg/mm² preferably 0.75-1.25 μg/mm².
Embodiment 11. The drug-eluting PCI of any one of Embodiments 1 to 10, wherein the weight of the CK applied on the blood vessel implant is more than 100 μg and less than 1000 μg.
Embodiment 12. The drug-eluting PCI of any one of Embodiments 1 to 11, wherein the blood vessel implant is a balloon.
Embodiment 13. The drug-eluting PCI of any one of Embodiments 1 to 12, further comprising a second layer under the first layer,
preferably the second layer comprises a second bioabsorbable polymer, more preferably the second bioabsorbable polymer comprises Polyvinylpyrrolidone (PVP) in a weight ratio of 80%-100%.
Embodiment 14. The drug-eluting PCI of Embodiment 13, wherein a thickness of the second layer ranging from 0.5 to 3 μm, from 1 to 3 μm, or from 0.5 to 1.5 μm.
Embodiment 15. The drug-eluting PCI of any one of Embodiments 1 to 14, wherein the first layer coated on the surface of the blood vessel implant is coated by the vacuum plasma spraying (VPS).
Embodiment 16. A method of preventing blood coagulation, comprising administering an effective amount of a ginsenoside Compound K (CK) to a subject in need.
Embodiment 17. Use of a ginsenoside Compound K (CK) in the manufacture a medicament for preventing blood coagulation.
Embodiment 18. A ginsenoside Compound K (CK) for use in a method of preventing blood coagulation.

### REFERENCE

[1] Zhang, J. H., Chung, T. D., and Oldenburg, K. R. (1999) A simple statistical parameter for use in evaluation and validation of high-throughput screening assays. J. Biomol. Screen. 4: 67-73.

1. A drug-eluting percutaneous coronary intervention (PCI), comprising a blood vessel implant,
wherein a surface of the blood vessel implant is coated with a first layer comprising ginsenoside Compound K (CK) and a first bioabsorbable polymer.

2. The drug-eluting PCI of claim 1, wherein the first bioabsorbable polymer comprises Poly-L-lactic acid (PLLA) and Poly(L-Lactide-co-ε-Caprolactone) (PLCL) in a weight ratio of 60%-80%:20%-40%.

3. The drug-eluting PCI of claim 1, wherein a thickness of the first layer ranging from 0.5 to 2 m, from 0.5 to 1.5 m, or from 0.8 to 1 m.

4. The drug-eluting PCI of claim 1, wherein the CK in the first bioabsorbable polymer is 0.1 to 5 μg/mm², 0.1-3 μg/mm², 0.1-1 μg/mm², 0.25-0.75 μg/mm² or 1-3 μg/mm².

5. The drug-eluting PCI of claim 1, wherein the blood vessel implant is a stent.

6. The drug-eluting PCI of claim 1, further comprising a second layer on top of the first layer,
preferably the second layer comprises a second bioabsorbable polymer, more preferably the second bioabsorbable polymer comprises Polyvinylpyrrolidone (PVP) in a weight ratio of 80%-100% based on the total weight of the second bioabsorbable polymer.

7. The drug-eluting PCI of claim 1, wherein a thickness of the second layer ranging from 0.5 to 3 μm, from 1 to 3 μm, or from 0.5 to 1.5 μm.

8. The drug-eluting PCI of claim 1, further comprising a third layer between the first layer and the second layer,
preferably the third layer comprises the CK and a third bioabsorbable polymer, more preferably the third bioabsorbable polymer comprises PLLA and PLCL in a weight ratio of 25%-35%:65%-85%.

9. The drug-eluting PCI of claim 1, wherein a thickness of the third layer ranging from 0.5 to 1.5 m.

10. The drug-eluting PCI of claim 1, wherein the CK in the third bioabsorbable polymer is 0.1 to 5 μg/mm², 0.5-2 μg/mm², preferably 0.75-1.25 μg/mm².

11. The drug-eluting PCI of claim 1, wherein the weight of the CK applied on the blood vessel implant is more than 100 μg and less than 1000 μg.

12. The drug-eluting PCI of claim 1, wherein the blood vessel implant is a balloon.

13. The drug-eluting PCI of claim 1, further comprising a second layer under the first layer,
preferably the second layer comprises a second bioabsorbable polymer, more preferably the second bioabsorbable polymer comprises Polyvinylpyrrolidone (PVP) in a weight ratio of 80%-100%.

14. The drug-eluting PCI of claim 13, wherein a thickness of the second layer ranging from 0.5 to 3 μm, from 1 to 3 μm, or from 0.5 to 1.5 μm.

15. The drug-eluting PCI of claim 1, wherein the first layer coated on the surface of the blood vessel implant is coated by the vacuum plasma spraying (VPS).

16. A method of preventing blood coagulation, comprising administering an effective amount of a ginsenoside Compound K (CK) to a subject in need.

17. (canceled)

18. (canceled)
